(19) United States
(12) Patent Application Publication
(10) Pub. No.: US 2012/0014903 A1
(43) Pub. Date: Jan. 19, 2012

(54) Cleaning Solution and Related Methods of Use

(76) Inventor: Thomas McCoy, St. Petersburg, FL (US)

(21) Appl. No.: 13/005,254

(22) Filed: Jan. 12, 2011

Related U.S. Application Data
(60) Provisional application No. 61/335,760, filed on Jan. 12, 2010.

(51) Int. Cl. A61L 9/01

(52) U.S. Cl. 424/76.9

(57) ABSTRACT

Disclosed is a substance and related methods for cleaning electronic devices which are to be used successively by different human operators.

### CROSS-REFERENCE TO RELATED APPLICATIONS

This application claims the benefit and priority of U.S. Prov. App. No. 61/335,760 (filed Jan. 12, 2010) entitled “Improved cleaning solution and related methods of use,” and said provisional application is hereby incorporated by reference in its entirety.

### STATEMENT REGARDING FEDERALLY SPONSORED RESEARCH OR DEVELOPMENT

None.

### BACKGROUND OF THE INVENTION

1. Field of Invention

The present invention is in the field of cleaning solutions and related methods of use.

2. Background of the Invention

Electronic devices are known to be used successively by human operators. For instance, this is the case with microphones used by successive performers on a stage. Such successive human interaction with the device invariably results in a contagion for communicating infectious organism, virus, odors, and/or other toxins between consecutive device operators. For example, a performer operating a microphone is often exposed to diseases deposited to the microphone by an earlier performer's hand, mouth, saliva and/or breath. Accordingly, there is a need for cleaning agents for electronic devices that are used successively by human operators.

Various liquid solutions are known that are capable of disinfecting/cleaning such electronic devices. For instance, Tidybio disinfecting solution (which contains the following active ingredients: (1) single and double stranded quaternary ammonium salt; and, (2) chlorhexadine acetate) has been used to clean electronic equipment because the solution does not have a measurable corrosive effect on stainless steel, copper, or aluminum. Such solutions are often sprayed onto the outer surface of the electronic device (see, e.g., U.S. Pat. Nos. 6,324,293 (issued Nov. 27, 2001) and 5,938,069 (issued Aug. 17, 1999) both teaching that electronic devices, namely microphones, may be cleaned via spraying a liquid cleaner onto the device). Nevertheless, liquid cleaning solutions and the associated methods of application have not been ideal for cleaning electronic devices since the liquid frequently may enter the interior of the device and interfere with the device's inner components. The identified drawback is particularly concerning for electronic devices, such as microphones, wherein the functioning components must be relatively unenclosed for ideal operation. For these reasons, there is still a need for improved cleaning agents and related methods for electronic devices that are used successively by different human operators.

Cleaning agents and related cleaning methods have been devised which may prevent the cleaning agent from entering the sensitive and internal areas of electronic devices. For example, U.S. Pat. No. 6,716,805 (issued Apr. 6, 2004) teaches the step of absorbing a liquid cleaning agent into a wipe, and then applying the cleaning agent to a target surface via the wipe because excess liquid is retained by the wipe. Although using a wipe reduces the amount of liquid solution that enters the sensitive areas of an electronic device by retaining excess cleaning solution within the wipe, such a method of cleaning is not satisfactory since additional effort is required to apply the cleaning agent into crevices or other hidden areas of the target surface. Accordingly, there is still a need for improved cleaning agents and associated application methods for electronic devices.

In addition to receiving pathogens, electronic devices that are used successively by different human operators may also receive deposits of material that are not infectious but nevertheless malodorous. Furthermore, the chemical components of cleaning agents applied to the surface of an electronic device may further contribute to the device's malodor. Malodorous electronic devices are particularly bothersome for applications wherein the device is frequently placed in the proximity of the operator's mouth and nose (e.g., microphone). As a result, there is a need for deodorizing agents for electronic devices that are cleaned or used successively by different human operators in close proximity to the operator's face.

A common method for alleviating odor is the addition of a perfume to a cleaning agent. See U.S. Pat. No. 6,716,805, col. 18:20 through col. 19:54. However, many of such cleaning agents are typically either not suitable for human contact or not of suitable coverage for electronic devices which are held in close proximity to a human mouth or nose. Furthermore, many deodorants are not sufficiently calibrated in potency to achieve maximum benefit, when diluted by inclusion within the cleaning agent. Therefore, there is a need for improved deodorizing agents and associated application methods for electronic devices that are used successively by different human operators in close proximity to the operator's face.

### SUMMARY OF THE INVENTION

It is an object of the present application to disclose cleaning agents and related methods for electronic devices that are used successively by different human operators in close proximity to the operator's face and that alleviates the problems associated with cleaning agents and methods which are heretofore known for the same purpose. In one non-limiting example, the disclosed cleaning agent may comprise: a quaternary ammonium salt, for example, didecyl dimethyl ammonium chloride (CAS: 7173-51-5); a chlorhexidine acetate, for example, chlorhexidine diacetate (CAS: 56-95-1); an emulsifier, for example, a TERGITOL™ NP-10 SURFACTANT (less than or equal to 97.0% Poly(oxy-1,2-ethanediyl), alpha-(4-nonylphenyl)-omegahydroxy-, branched (CAS: 127087-87-0), greater than or equal to 3.0% Poly(ethylene oxide) (CAS: 25322-68-3), and greater than or equal to 2.0% Dinonylphenyl polyoxyethylene (CAS: 9014-93-1)); a conditioner or wetting agent, for example, glycerine (CAS: 56-81-5); a deodorant, for example, a cherry flavor/scent compound (primarily comprised of phenylethyl alcohol, benzyl alcohol, vanillin, and propylene glycol); and purified water. The disclosed composition preferably kills 99.99% of germs, is quick-drying, and substantially eliminates or masks malodor. Further disclosed is a method of cleaning an electronic device comprising the step of: (1) collecting an amount of the cleaning agent in a hand; (2) applying the cleaning agent to a target region of a surface, for example, the receiver or head of a microphone; and, (3) allowing the cleaning agent to quickly evaporate.

It is further an object of the present invention to disclose a cleaning agent for devices which are successively used by different human operators in close proximity to the operator's face which will not seep into the interior of an electronic device without the need for a wipe or wash cloth. To accomplish these objectives, the disclosed substance is preferably: foamable; quick drying; and not sprayed directly onto an electronic device, but rather manually applied thereto.

It is yet another object of the present invention to disclose a cleaning agent for devices which are successively used by different human operators in close proximity to the operator's face which will mask odor from the component chemicals and from the electronic device via a deodorant in dilute quantities. To accomplish these objectives, a particular flavor or perfume is suitably mixed with the disclosed substance, which flavor or perfume is more particularly adapted to overcome intense malodor despite significant dilution than those previously used for electronic device cleaning agents.

### BRIEF DESCRIPTION OF THE FIGURES

The manner in which these objectives and other desirable characteristics can be obtained is better explained in the following description and attached figures in which:

FIG. 1 is a chemical structure of didecyl dimethyl ammonium chloride.

FIG. 2 is a chemical structure of chlorhexidine diacetate.

FIG. 3 is a chemical structure of glycerin.

FIG. 4 is a flow diagram illustrating the steps associated with cleaning an electronic device using the disclosed substance.

It is to be noted, however, that the appended figures illustrate only typical embodiments of this invention, and therefore, are not to be considered limiting of its scope, for the invention may admit to other equally effective embodiments that will be appreciated by those reasonably skilled in the relevant arts. Also, figures are not necessarily made to scale.

### DETAILED DESCRIPTION OF PREFERRED EMBODIMENTS

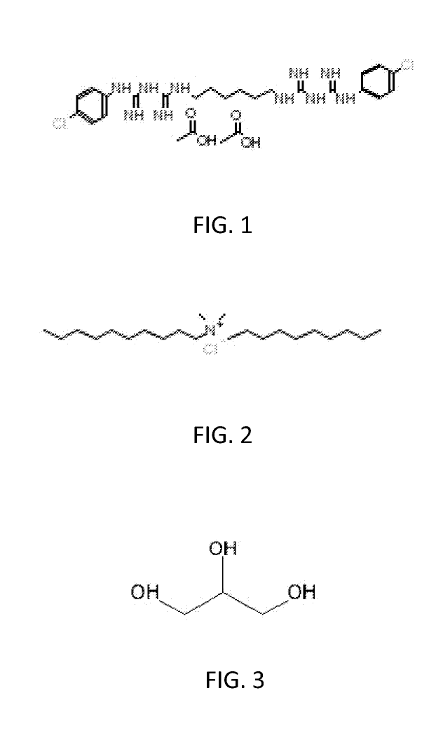

In general, a preferred embodiment of the present application may be a cleaning agent comprising: a quaternary ammonium salt, for example, 99.3% pure didecyl dimethyl ammonium chloride (CAS: 7173-51-5) (EPA Reg. No. 1117-62) (see FIG. 1); a chlorhexidine acetate, for example, 80% pure chlorhexidine diacetate (CAS: 56-95-1) (EPA Reg. No. 1839-135) (see FIG. 2); an emulsifier, for example, a TERGITOL™ NP-10 SURFACTANT (less than or equal to 97.0% Poly(oxy-1,2-ethanediyl), alpha-(4-nonylphenyl)-omegahydroxy-, branched (CAS: 127087-87-0), greater than or equal to 3.0% Poly(ethylene oxide) (CAS: 25322-68-3), and greater than or equal to 2.0% Dinonylphenyl polyoxyethylene (CAS: 9014-93-1)); a conditioner or wetting agent, for example, glycerin (CAS: 56-81-5) (see FIG. 3); a deodorant, for example, a cherry flavor/scent compound (primarily comprised of phenylethyl alcohol, benzyl alcohol, vanillin, and propylene glycol); and a solvent or carrier, for example, purified water. In formulation, a preferred embodiment is ideally: approx. 0.18% quaternary ammonium salt, by weight; approx. 0.22% chlorhexidine acetate, by weight; approx. 0.16% emulsifier, by weight; approx. 0.16% conditioner, by weight; approx. 0.89% deodorant, by weight; and, approx. 98.50% solvent, by weight. Although given the ideal formulation above, another preferable formulation features: a quaternary ammonium salt in a range of about 0.162% to 0.200%, by weight; chlorhexidine acetate in a range of about 0.162% to 0.244%, by weight; emulsifier in a range of about 0.140% to 0.180%, by weight; a conditioner in a range of about 0.140% to 0.180%, by weight; a deodorant in a range of about 0.710% to 0.890%, by weight; and, a solvent in a range of about 99.00% to 98.00%, by weight.

The above disclosed formulation is important in regard to the amount of deodorant provided to the cleaning agent. As mentioned above, the deodorants provided to the cleaning agents of the prior art must be particularly concentrated to mask malodor. The presently disclosed embodiment is superior to the prior art in that it merely requires the presence of a deodorant in the quantity in a range of about 0.710% to 0.890%, by weight. The recited minimal amount of deodorant is further enhanced with respect to its malodor-masking properties via the use of a cherry scent/flavor, which broadcasts its malodor-masking scent in a manner which is the olfactory equivalent to disruptions in sound-waves that result in “square wavelengths.”

The above identified preferable embodiment may be prepared by the following non-limiting example. First, the appropriate component materials must be obtained. A quaternary ammonium salt, for example, didecyl dimethyl ammonium chloride, may be prepared according to the disclosure of U.S. Pat. No. 4,666,896 (issued May 19, 1987). A chlorhexidine acetate, for example, chlorhexidine diacetate, may be prepared according to the disclosure of U.S. Pat. No. 3,836,669 (issued Sep. 17, 1974). An emulsifier, for example, TERGITOL™ NP-10 SURFACTANT may be purchased from Dow Chemical Company or the Stepan Company of Northfield, Ill. A conditioner, for example, glycerin, may be prepared according to the disclosure of U.S. Pat. No. 601,603 (issued Mar. 29, 1898). A deodorant, for example, cherry flavor compound, may be purchased from The Berjie Co. of Bloomfield, N.J. or Handyware Ltd. of China. A solvent, for example, pure water, may be obtained by boiling water and condensing the vapor. Second, the component materials may be combined into a preferred embodiment as follows. Initially, the solvent may be used to dissolve the appropriate amounts of the quaternary ammonium salt, chlorhexidine acetate, conditioner, and deodorant. Next, the solution may be foamed via the addition of the emulsifier and mixing thoroughly with a gas or according to similar steps as those disclosed in U.S. Pat. No. 4,915,394 (issued Apr. 10, 1990). Finally, the mixture may be placed in a container or dispenser.

Physically and chemically, a preferred embodiment thus disclosed features the following properties: 0 to 0.2% volatility by volume; a specific gravity of 0.98; a 97 to 98% evaporation rate; a boiling point of 100 degrees centigrade; a freezing point of 0 degrees centigrade; a pH of 6.65 to 6.77; and, 100% solubility in water. One preferred embodiment further produces no corrosive effects on stainless steel, copper or aluminum, but has light corrosive effects on carbon steel. After a 0.5 minute period, the average killing logarithm of the proffered embodiment is greater than 5.00 for Staphylococcus aureus, Escherichia coli, P. aeruginasa; for Blastomyces albicans the average killing logarithm was greater than 4.00 over the same period. After a 3.0 minute period the average killing logarithm for bacteria and influenza virus on a fabric, object, or skin is greater than 1.00. The median lethal dose of the disclosed substance in mice is preferably more than 5000 mg/kg, while the maximum harmless dose is suitably 1000 mg/kg. After placement of the disclosed substance on damaged or intact rabbit skin and rabbit vaginal mucosa, the stimulation intensity preferably indicated a light level. The substance may cause moderate eye irritation in humans, but may be removed by rinsing the eye with water for 15 to 20 minutes. In other words, the disclosed composition preferably kills 99.99% of germs, is relatively non-toxic to humans, not damaging of electronic devices, is quick-drying, and substantially eliminates or masks malodor.

The disclosed substance represents a significant advantage over the prior art. First, the substance is preferably foam whereby the substance remains relatively confined to the application area when directed toward an electronic device. Second, the substance typically vaporizes before the foam can re-liquefy and thereby the reliquified cleaner cannot pass within the device to interfere with its internal components. Finally, the square wavelength deodorant is significantly better at masking malodor than substances previously provided to electronic device cleaning agents.

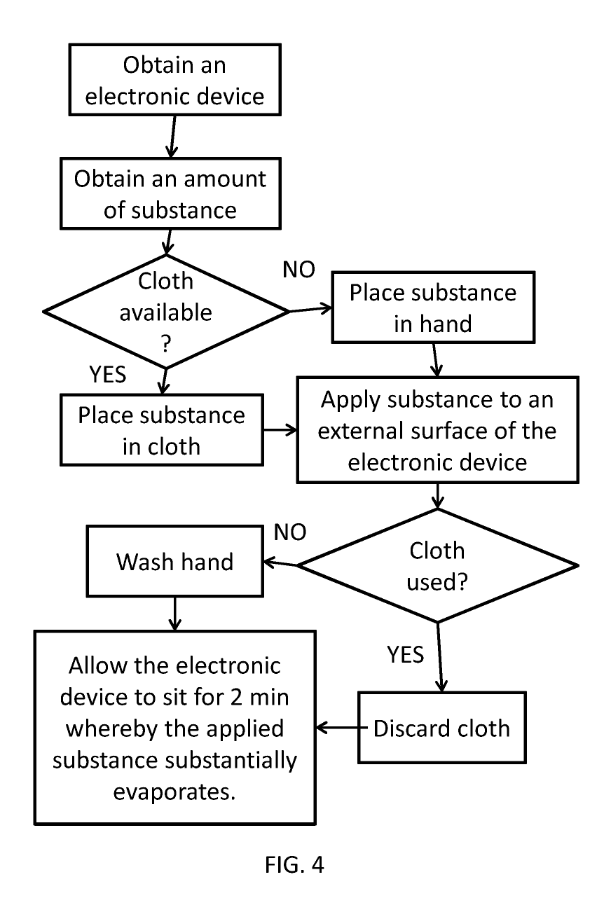

FIG. 4 depicts the flow of a suitable method of cleaning an electronic device. As seen in the figure, the suitable method comprises the steps of: (1) unplugging an electronic device; (2) collecting an amount of the disclosed cleaning agent in a hand or alternatively in a cloth; (3) applying the cleaning agent via the hand or cloth to a target region of an exterior surface on the device (for example, the receiver or head of a microphone) via spreading the substance over the surface; (4) allowing the cleaning agent to remain on the surface for 2 minutes, whereby the solution substantially evaporates from the surface; and, washing the hand or discarding the cloth. The recited method is particularly appropriate for circumstances wherein: the electronic device is a microphone and the exterior surface of the device is the head of the microphone; the electronic device is a headphone; the electronic device is a telephone; the electronic device is audio electronic equipment. Although discussed in connection with electronic devices, it should be noted that the disclosed substance may be used on, according to the above recited method, non-electronic devices, e.g., business, household, sporting, and entertainment objects including but not limited to door knobs/handles, bathroom and kitchen plumbing fixtures, automobile steering wheels and door or glove compartment latches, and boating steering devices and throttles, musical instruments, and the like.

The disclosed method represents an advancement over methods previously known for cleaning microphones because the method applies the substance to electronic devices in a way that avoids the drawbacks listed in the background of the invention.

In summary, what is disclosed may be a cleaning agent comprising: a quaternary ammonium salt, chlorhexidine acetate, an emulsifier, a conditioner, a square wave-length deodorant, and a solvent. What is also disclosed is the substance of the preceding sentence wherein the quaternary ammonium salt is didecyl dimethyl ammonium chloride, the chlorhexidine acetate is chlorhexidine diacetate, the emulsifier, is TERGITOL™ NP-10 SURFACTANT, the conditioner is glycerin, the deodorant is cherry flavor/scent compound, and the solvent is purified water. What is further disclosed may be a method of cleaning a microphone, the method comprising the steps of unplugging the microphone, spreading the disclosed substance over the head of the microphone, and allowing the substance to evaporate from the head of the microphone.

It should be noted that FIGS. 1 through 4 are of illustrative importance only. In other words, the flow of methods and descriptions of the present application should not be construed as limiting of the subject matter in this application. The substances and methods discussed hereby are susceptible to modification without changing the overall concept of the disclosure. For example, the relative amounts of the component chemicals may be varied according to the above disclosure. For another example, the methods recited hereby may be accomplished with fewer or additional steps. Additional modifications may become apparent to one skilled in the art after reading this disclosure.

What is disclosed may be:

A foaming electronic device cleaning substance comprising:
a quaternary ammonium salt present in a range of about 0.162% to 0.200%, by weight;
a chlorhexidine acetate in a range of about 0.162% to 0.244%, by weight;
an emulsifier in a range of about 0.140% to 0.180%, by weight;
a conditioner in a range of about 0.140% to 0.180%, by weight;
a deodorant in a range of about 0.710% to 0.890%, by weight; and,
a solvent in a range of about 99.00% to 98.00%, by weight.

A foaming electronic device cleaning substance comprising:
a quaternary ammonium salt; a chlorhexidine acetate; an emulsifier; a conditioner; a deodorant in a range of about 0.710% to 0.890%, by weight; and, a solvent.

A method of fabricating a foaming cleaner comprising the steps of:
dissolving a quaternary ammonium salt, a chlorhexidine acetate, a conditioner, and a deodorant into a solvent; and,
mixing the resultant solution with a gas in the presence of an emulsifier.

A method of cleaning an electronic device comprising the steps of:
obtaining a foam cleaning agent comprising a quaternary ammonium salt, a chlorhexidine acetate, an emulsifier, a conditioner, a deodorant, and a solvent;
placing an amount of the cleaning agent into the palm of a hand;
directing, via the hand, the substance toward an external surface of the electronic device;
allowing the foam to sit for about two-minutes.

I claim:

1. A method of cleaning an electronic device comprising the steps of:
obtaining a foam cleaning agent comprising a quaternary ammonium salt, a chlorhexidine acetate, an emulsifier, a conditioner, a deodorant, and a solvent;
placing an amount of the cleaning agent into the palm of a hand;
directing, via the hand, the substance toward an external surface of the electronic device;
allowing the foam to sit for about two-minutes.
